(19) United States
(12) Patent Application Publication
(10) Pub. No.: US 2009/0082836 A1
(43) Pub. Date: Mar. 26, 2009

(54) Methods of applying phototherapy

(76) Inventor: Daniel Schell, Los Gatos, CA (US)

Correspondence Address: PATENT LAW & VENTURE GROUP, 2424 S.E. BRISTOL, SUITE 300, NEWPORT BEACH, CA, 92660, US

(21) Appl. No.: 11/903,069

(22) Filed: Sep. 20, 2007

(51) Int. Cl. A61N 5/06

(52) U.S. Cl. 607/88

(57) ABSTRACT

A method of applying phototherapy to a skin surface region of the body improves, relieves or eliminates disease and undesirable conditions. In many cases damage is reversed. The method includes contacting the skin surface region with a plurality of light emitters, the light emitters positioned so as to generate an approximately uniform light emission over the skin surface region; emitting plural sequenced light emissions from the light emitters, wherein each one of the plural light emissions extends over a selected time duration, is pulsed at a selected pulse rate, and is centered on a selected wavelength of light; and emitting the light emissions alternately in the red region of the light spectrum and the infrared region of the light spectrum.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not applicable.

### REFERENCE TO A “MICROFICHE APPENDIX”

Not applicable.

### BACKGROUND OF THE INVENTION

1. Field of the Present Disclosure

This disclosure relates generally to light emission phototheraphy for the improvement of medical conditions, and more particularly a method that uses LEDs mounted on a pad for intimate contact with a skin surface and the use of both red and infrared emissions in an alternating protocol.

2. Description of Related Art Including Information Disclosed Under 37 CFR 1.97 and 1.98

Relevant prior art to the present invention includes: Streeter, U.S. Pat. No. 6,312,451, Pecukonis, U.S. Pat. No. 6,471,716, Pederson et al, U.S. Pat. No. 6,875,225, Trauner et al, U.S. Pat. No. 5,913,884, Van Zuylen et al, U.S. Pat. No. 6,221,095, Groves, U.S. Pat. No. 4,011,575, Sullivan, U.S. Pat. No. 6,896,693, and Mendes et al, U.S. Pat. No. 5,259,380. For example Mendes et al teaches an array of light emitting diodes presenting light onto a dermal surface with pulsed output in the red or infrared bandwidth and with durations of 5 to 10 minutes and repeating a limited number of times daily. Sullivan teaches a similar method for relaxation and detoxification of an organism, and for stimulating the healing process and elimination of pain and inflammation. Trauner et al teaches a method for the elimination of microphages and myofibrablasts. Pecukonis teaches a device similar to that of the present invention utilizing a field of emitters and is directed to treating insect bite and headache. Streater teaches the use of light in the bandwidth between 630 and 904 nm. The above eight references are incorporated herein by reference for their teaching of light emitters, power supplies with pulse forming capabilities and methods of treatment.

The related art described above discloses phototherapy methods and the apparatuses of applying such methods. However, the prior art fails to disclose the use of certain critical pulse rates related to specific therapy and the alternating of red and infrared emissions over a sequence of emissions for a set period of therapy. The present disclosure distinguishes over the prior art providing heretofore unknown advantages as described in the following summary.

### BRIEF SUMMARY OF THE INVENTION

This disclosure teaches certain benefits in construction and use which give rise to the objectives described below.

The present invention is a phototherapy method of applying light energy to a skin surface region of the body which improves, relieves or eliminates disease and undesirable conditions. In many cases damage is reversed. The method includes contacting the skin surface region with a plurality of light emitters, the light emitters positioned so as to generate an approximately uniform light emission over the skin surface region; emitting plural sequenced light emissions from the light emitters, wherein each one of the plural light emissions extends over a selected time duration, is pulsed at a selected pulse rate, and is centered on a selected wavelength of light; and emitting the light emissions alternately in the red region of the light spectrum and the infrared region of the light spectrum.

A primary objective inherent in the above described apparatus and method of use is to provide advantages not taught by the prior art.

Another objective is to provide a simple method of applying phototherapy to a surface of the body.

A further objective is to provide a protocol including timing, pulse rate, light wavelength and sequencing that results in improvement to or cessation of detrimental effects in the skin and other organs.

Other features and advantages of the present invention will become apparent from the following more detailed description, taken in conjunction with the accompanying drawings, which illustrate, by way of example, the principles of the presently described apparatus and method of its use.

### DESCRIPTION OF THE DRAWING

Illustrated in the accompanying drawing(s) is at least one of the best mode embodiments of the present invention In such drawing(s):

FIG. 1 is a schematic diagram of the apparatus of the present invention.

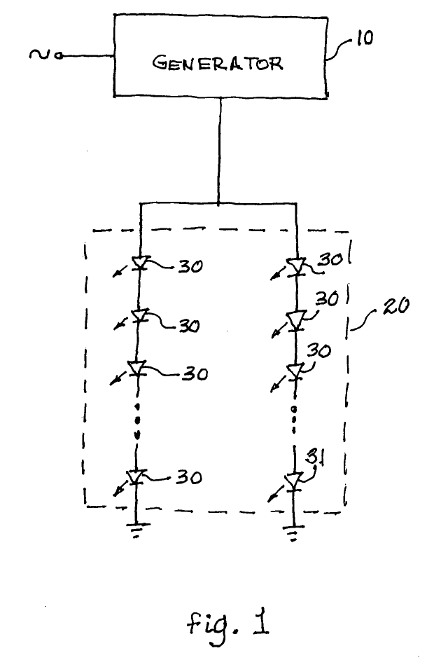

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications to what is described herein without departing from its spirit and scope. Therefore, it must be understood that what is illustrated is set forth only for the purposes of example and that it should not be taken as a limitation in the scope of the present apparatus and method of use.

Described now in detail is a method for applying phototherapy to the skin surface of a region of the human body, for improving a problem condition and reversing damage to both the skin as well as organs below the region being treated. The method includes the steps of: contacting the skin surface region with a plurality of light emitters simultaneously, positioning the light emitters so as to generate an approximately uniform light emission over a selected skin surface region, emitting plural sequenced light emissions from the light emitters, wherein each one of the plural light emissions extends over a selected time duration, is pulsed at a selected pulse rate, and is centered on a selected wavelength of light; and emitting the light emissions alternately in the red region of the light spectrum and the infrared region of the light spectrum. It has been found to be critically important to alternate between these two regions of the light spectrum for a variety of reasons and this is an important novelty of the present invention. We have found advantage to using time durations of not less then one minute and not exceeding twenty minutes depending on the condition being treated and the objective of the treatment. We have found advantage to using pulse rates between 10 and 20,000 pulses per second, again, depending on the condition and objective. The pulses are square waves with essentially vertical leading and trailing edges. We have found advantage to using light wavelengths that are centered in one of the red and infrared portions of the light spectrum as stated above, depending, once again, on the condition being treated and the objective. Clearly, no single treatment is efficacious in phototherapy treatments so that, in almost all cases, a regimen of treatments spread over days, weeks and months, depending on objective and case improvement is usually called for. We have discovered that: 11, 73, 146, 292, 585, 1168, 2336, 4672, 660, 690, 727, 787, 800, 880, 1500, 1550, 2008, 2128, 9344, and pulses per second are the rates that produce effective treatments and meet the herein described objectives most efficaciously.

The apparatus for administrating the phototherapy is a square wave generator circuit 10 able to output variable driver pulse rates, with timers and voltage output control. Such a circuit is well known in industry. Light is applied by a flexible pad 20 of approximately 8 inches square. Placed within the fabric of the pad 20 are a field of light emitting diodes (LEDs) 30 that are placed in electrical series-parallel and which project their energy normal to the surface of the pad 20. The field of LEDs 30 comprise both red light emitters and also infrared light emitters. Since the protocol for emission of light from the pad 20 calls for alternating red and infrared, the LEDs 30 are spaced alternately on the pad 20. The pad 20 and its LEDs 30 is placed onto the skin surface with the LEDs 30 in contact with the skin. Light energy from the LEDs 30 is transmitted into and through the skin surface into the tissue below. When the red LEDs 30 are activated, they are all energized at once and similarly with the infrared LEDs 30. The result is a relatively uniform field of light applied over a target surface area on any part of the body.

In using the above variables and variable ranges specified we have found that normally aged skin, sun damaged skin, and minor discolored skin is improved by applying the following specific protocol:
a) a red emission at 292 hz pulse rate for 2 minutes;
b) an infrared emission at 292 hz pulse rate for 1 minute;
c) a red emission at 584 hz pulse rate for 6 minutes;
d) an infrared emission at 584 hz pulse rate for 1 minute;
e) a red emission at 2336 hz pulse rate for 6 minutes; and
f) an infrared emission at 2336 hz pulse rate for 4 minutes.

In using the above variables and the variable ranges specified we have found that thinned skin, light sensitive skin, newly damaged skin, and dry skin is improved by applying the following specific protocol:
a) a red emission at 292 hz pulse rate for 4 minutes;
b) an infrared emission at 11 hz pulse rate for 1 minute;
c) a red emission at 292 hz pulse rate for 4 minutes;
d) an infrared emission at 73 hz pulse rate for 1 minute;
e) a red emission at 292 hz pulse rate for 4 minutes;
f) an infrared emission at 11 hz pulse rate for 1 minute;
g) a red emission at 292 hz pulse rate for 4 minutes; and
h) an infrared emission at 73 hz pulse rate for 1 minute.

In using the above variables and the variable ranges specified we have found that older damaged skin, bruised skin, and inflamed skin is improved by applying the following specific protocol:
a) a red emission at 73 hz pulse rate for 4 minutes;
b) an infrared emission at 11 hz pulse rate for 1 minute;
c) a red emission at 146 hz pulse rate for 4 minutes;
d) an infrared emission at 11 hz pulse rate for 1 minute;
e) a red emission at 584 hz pulse rate for 5 minutes; and
f) an infrared emission at 584 hz pulse rate for 5 minutes.

In using the above variables and the variable ranges specified we have found that skin having redness, and skin with broken capillaries is improved by applying the following specific protocol:
a) a red emission at 146 hz pulse rate for 4 minutes;
b) an infrared emission at 146 hz pulse rate for 1 minute;
c) a red emission at 584 hz pulse rate for 4 minutes;
d) an infrared emission at 584 hz pulse rate for 1 minute;
e) a red emission at 1168 hz pulse rate for 5 minutes; and
f) an infrared emission at 2336 hz pulse rate for 5 minutes.

In using the above variables and the variable ranges specified we have found that hyper-pigmented skin, skin with dark spots, and sun spotted skin is improved by applying the following specific protocol:
a) a red emission at 584 hz pulse rate for 3 minutes;
b) an infrared emission at 584 hz pulse rate for 2 minutes;
c) a red emission at 2336 hz pulse rate for 3 minutes;
d) an infrared emission at 2336 hz pulse rate for 2 minutes;
e) a red emission at 4672 hz pulse rate for 3 minutes;
f) an infrared emission at 4672 hz pulse rate for 2 minutes;
g) a red emission at 9344 hz pulse rate for 1 minutes; and
h) an infrared emission at 9344 hz pulse rate for 4 minute.

In using the above variables and the variable ranges specified we have found that chronic injuries, carpel tunnel, older skin damage is improved by applying the following specific protocol:
a) a red emission at 73 hz pulse rate for 2 minutes;
b) an infrared emission at 73 hz pulse rate for 3 minutes;
c) a red emission at 146 hz pulse rate for 2 minutes;
d) an infrared emission at 146 hz pulse rate for 3 minutes;
e) a red emission at 584 hz pulse rate for 5 minutes; and
f) an infrared emission at 584 hz pulse rate for 5 minutes.

In using the above variables and the variable ranges specified we have found that chronic injuries, bone/joint, inflamed skin, skin with redness, scraped or abraded skin and older injuries respond by applying the following specific protocol:
a) a red emission at 73 hz pulse rate for 1 minute;
b) an infrared emission at 73 hz pulse rate for 4 minutes;
c) a red emission at 584 hz pulse rate for 2 minutes;
d) an infrared emission at 584 hz pulse rate for 3 minutes;
e) a red emission at 1168 hz pulse rate for 5 minutes; and
f) an infrared emission at 1168 hz pulse rate for 5 minutes.

In using the above variables and the variable ranges specified we have found that skin stretch marks, new scars, and new injuries respond by applying the following protocol:
a) a red emission at 146 hz pulse rate for 2 minutes;
b) an infrared emission at 292 hz pulse rate for 3 minutes;
c) a red emission at 292 hz pulse rate for 2 minutes;
d) an infrared emission at 584 hz pulse rate for 3 minutes;
e) a red emission at 2336 hz pulse rate for 5 minutes; and
f) an infrared emission at 4672 hz pulse rate for 5 minutes.

In using the above variables and the variable ranges specified we have found that deep skin stretch marks, old scars, tendons, and large muscle groups respond positively by applying the following specific protocol:
a) a red emission at 146 hz pulse rate for 1 minute;
b) an infrared emission at 146 hz pulse rate for 3 minutes;
c) a red emission at 584 hz pulse rate for 1 minute;
d) an infrared emission at 584 hz pulse rate for 3 minutes;
e) a red emission at 1168 hz pulse rate for 2 minutes;
f) an infrared emission at 1168 hz pulse rate for 4 minutes;
g) a red emission at 4672 hz pulse rate for 2 minutes; and
h) an infrared emission at 4672 hz pulse rate for 4 minutes.

In using the above variables and the variable ranges specified we have found that hair growth is promoted by applying the following specific protocol:
a) a red emission at 73 hz pulse rate for 4 minutes;
b) an infrared emission at 292 hz pulse rate for 1 minute;
c) a red emission at 584 hz pulse rate for 3 minutes;
d) an infrared emission at 584 hz pulse rate for 2 minutes;
e) a red emission at 2336 hz pulse rate for 5 minutes; and
f) an infrared emission at 73 hz pulse rate for 5 minutes.

In using the above variables and the variable ranges specified we have found that pathogens may be destroyed by applying the following specific protocol:
a) a red emission at 660 hz pulse rate for 3 minute;
b) an infrared emission at 690 hz pulse rate for 3 minutes;
c) a red emission at 727 hz pulse rate for 3 minute;
d) an infrared emission at 787 hz pulse rate for 3 minutes;
e) a red emission at 800 hz pulse rate for 3 minutes;
f) an infrared emission at 880 hz pulse rate for 3 minutes;
g) a red emission at 1500 hz pulse rate for 3 minutes; and
h) an infrared emission at 1550 hz pulse rate for 3 minutes.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. A method of applying phototherapy to a skin surface region of a body, for improving the condition of the region of the body and reversing damage, comprising the steps of:
a) contacting the skin surface region with a plurality of light emitters, the light emitters positioned so as to generate an approximately uniform light emission over the skin surface region;
b) emitting plural sequenced light emissions from the light emitters, wherein each one of the plural light emissions extends over a selected time duration, is pulsed at a selected pulse rate, and is centered on a selected wavelength of light; and
c) emitting the light emissions alternately in the red region of the light spectrum and the infrared region of the light spectrum.

2. The method of claim 1 wherein each one of the time durations is between 1 and 20 minutes.

3. The method of claim 1 wherein each one of the pulse rates is between 10 and 20,000 pulses per second.

4. The method of claim 1 wherein each said wavelength of light is centered in one of the red and infrared portions of the light spectrum.

5. The method of claim 3 wherein the pulse rate is one of: 11, 73, 146, 292, 585, 1168, 2336, 4672, 660, 690, 727, 787, 800, 880, 1500, 1550, 2008, 2128, 9344 pulses per second.

7. The method of claim 1 wherein each one of the time durations is between 1 and 20 minutes; and each one of the pulse rates is between 10 and 20,000 pulses per second; and the wavelength of light is centered in one of the red and infrared portion of the light spectrum.

8. The method of claim 7 comprising 6 light emitting sequences including, in sequential order:
g) a red emission at 292 hz pulse rate for 2 minutes;
h) an infrared emission at 292 hz pulse rate for 1 minute;
i) a red emission at 584 hz pulse rate for 6 minutes;
j) an infrared emission at 584 hz pulse rate for 1 minute;
k) a red emission at 2336 hz pulse rate for 6 minutes; and
l) an infrared emission at 2336 hz pulse rate for 4 minutes;
whereby, normally aged skin, sun damaged skin, and minor discolored skin is improved.

9. The method of claim 7 comprising 8 light emitting sequences including, in sequential order:
i) a red emission at 292 hz pulse rate for 4 minutes;
j) an infrared emission at 11 hz pulse rate for 1 minute;
k) a red emission at 292 hz pulse rate for 4 minutes;
l) an infrared emission at 73 hz pulse rate for 1 minute;
m) a red emission at 292 hz pulse rate for 4 minutes;
n) an infrared emission at 11 hz pulse rate for 1 minute;
o) a red emission at 292 hz pulse rate for 4 minutes; and
p) an infrared emission at 73 hz pulse rate for 1 minute;
whereby, thinned skin, light sensitive skin, cosmetic damaged skin, and dry skin is improved.

10. The method of claim 7 comprising 6 light emitting sequences including, in sequential order:
g) a red emission at 73 hz pulse rate for 4 minutes;
h) an infrared emission at 11 hz pulse rate for 1 minute;
i) a red emission at 146 hz pulse rate for 4 minutes;
j) an infrared emission at 11 hz pulse rate for 1 minute;
k) a red emission at 584 hz pulse rate for 5 minutes; and
l) an infrared emission at 584 hz pulse rate for 5 minutes;
whereby, older damaged skin, bruised skin, and inflamed skin is improved.

11. The method of claim 7 comprising 6 light emitting sequences including, in sequential order:
g) a red emission at 146 hz pulse rate for 4 minutes;
h) an infrared emission at 146 hz pulse rate for 1 minute;
i) a red emission at 584 hz pulse rate for 4 minutes;
j) an infrared emission at 584 hz pulse rate for 1 minute;
k) a red emission at 1168 hz pulse rate for 5 minutes; and
l) an infrared emission at 2336 hz pulse rate for 5 minutes;
whereby, skin having redness, and skin with broken capillaries is improved.

12. The method of claim 7 comprising 8 light emitting sequences including, in sequential order:
i) a red emission at 584 hz pulse rate for 3 minutes;
j) an infrared emission at 584 hz pulse rate for 2 minutes;
k) a red emission at 2336 hz pulse rate for 3 minutes;
l) an infrared emission at 2336 hz pulse rate for 2 minutes;
m) a red emission at 4672 hz pulse rate for 3 minutes;
n) an infrared emission at 4672 hz pulse rate for 2 minutes;
o) a red emission at 9344 hz pulse rate for 1 minutes; and
p) an infrared emission at 9344 hz pulse rate for 4 minute;
whereby, hyper-pigmented skin, skin with dark spots, and sun spotted skin is improved.

13. The method of claim 7 comprising 6 light emitting sequences including, in sequential order:
g) a red emission at 73 hz pulse rate for 2 minutes;
h) an infrared emission at 73 hz pulse rate for 3 minutes;
i) a red emission at 146 hz pulse rate for 2 minutes;
j) an infrared emission at 146 hz pulse rate for 3 minutes;
k) a red emission at 584 hz pulse rate for 5 minutes; and
l) an infrared emission at 584 hz pulse rate for 5 minutes;
whereby, chronic injuries, carpel tunnel, older skin damage is improved.

14. The method of claim 7 comprising 6 light emitting sequences including, in sequential order:
g) a red emission at 73 hz pulse rate for 1 minute;
h) an infrared emission at 73 hz pulse rate for 4 minutes;
i) a red emission at 584 hz pulse rate for 2 minutes;
j) an infrared emission at 584 hz pulse rate for 3 minutes;
k) a red emission at 1168 hz pulse rate for 5 minutes; and
l) an infrared emission at 1168 hz pulse rate for 5 minutes;
whereby chronic injuries, bone/joint, inflamed skin, skin with redness, scraped or abraded skin and older injuries are improved.

15. The method of claim 7 comprising 6 light emitting sequences including, in sequential order:
g) a red emission at 146 hz pulse rate for 2 minutes;
h) an infrared emission at 292 hz pulse rate for 3 minutes;
i) a red emission at 292 hz pulse rate for 2 minutes;
j) an infrared emission at 584 hz pulse rate for 3 minutes;
k) a red emission at 2336 hz pulse rate for 5 minutes; and
l) an infrared emission at 4672 hz pulse rate for 5 minutes;
whereby, skin stretch marks, new scars, and new injuries are reduced.

16. The method of claim 7 comprising 8 light emitting sequences including, in sequential order:
i) a red emission at 146 hz pulse rate for 1 minute;
j) an infrared emission at 146 hz pulse rate for 3 minutes;
k) a red emission at 584 hz pulse rate for 1 minute;
l) an infrared emission at 584 hz pulse rate for 3 minutes;
m) a red emission at 1168 hz pulse rate for 2 minutes;
n) an infrared emission at 1168 hz pulse rate for 4 minutes;
o) a red emission at 4672 hz pulse rate for 2 minutes; and
p) an infrared emission at 4672 hz pulse rate for 4 minutes;
whereby, deep skin stretch marks, and old scars are reduced.

17. The method of claim 7 comprising 6 light emitting sequences including, in sequential order:
g) a red emission at 73 hz pulse rate for 4 minutes;
h) an infrared emission at 292 hz pulse rate for 1 minute;
i) a red emission at 584 hz pulse rate for 3 minutes;
j) an infrared emission at 584 hz pulse rate for 2 minutes;
k) a red emission at 2336 hz pulse rate for 5 minutes; and
l) an infrared emission at 73 hz pulse rate for 5 minutes;
whereby, hair growth is promoted.

18. The method of claim 7, comprising 4 light emitting sequences including, in sequential order:
a) a red emission at 9344 hz pulse rate for 5 minutes;
b) an infrared emission at 9344 hz pulse rate for 5 minutes;
c) a red emission at 9344 hz pulse rate for 5 minutes; and
d) an infrared emission at 9344 hz pulse rate for 5 minutes;
whereby, relaxation is improved.

19. The method of claim 7, comprising 2 light emitting sequences including, in sequential order:
a) a red emission at 14 hz pulse rate for 10 minutes; and
b) an infrared emission at 14 hz pulse rate for 10 minutes;
whereby, wellness is improved.

20. The method of claim 7 comprising 8 light emitting sequences including, in sequential order:
i) a red emission at 660 hz pulse rate for 3 minute;
j) an infrared emission at 690 hz pulse rate for 3 minutes;
k) a red emission at 727 hz pulse rate for 3 minute;
l) an infrared emission at 787 hz pulse rate for 3 minutes;
m) a red emission at 800 hz pulse rate for 3 minutes;
n) an infrared emission at 880 hz pulse rate for 3 minutes;
o) a red emission at 1500 hz pulse rate for 3 minutes; and
p) an infrared emission at 1550 hz pulse rate for 3 minutes;
whereby, pathogens are destroyed.
